(19) United States
(12) Patent Application Publication
(10) Pub. No.: US 2024/0277515 A1
(43) Pub. Date: Aug. 22, 2024

(54) Massage Stone Heating and Cooling Device

(71) Applicant: Shelle Hagen, Moorhead, MN (US)

(72) Inventor: Shelle Hagen, Moorhead, MN (US)

(21) Appl. No.: 18/418,439

(22) Filed: Jan. 22, 2024

Related U.S. Application Data
(60) Provisional application No. 63/446,144, filed on Feb. 16, 2023.

(51) Int. Cl. A61F 7/02; A61F 7/00; A61H 7/00; H05B 3/20

(52) U.S. Cl. CPC A61F 7/0241; A61F 7/007; A61H 7/003; H05B 3/20; A61F 2007/0093; A61F 2007/0094; A61H 2201/0207; A61H 2201/0214; A61H 2201/169

(57) ABSTRACT

A massage stone heating and cooling device is disclosed for heating and cooling at least one massage stone. The device heats and cools massage stones via a waterless heating and cooling element. The massage stone heating and cooling device comprises a housing with a lid that offers more efficient temperature regulation. Further, the housing comprises a digital temperature reader and control which keeps the stones from getting too hot for the skin. The housing also comprises a silicone mat insert that reduces noise and movement.

### CROSS-REFERENCE TO RELATED APPLICATION

The present application claims priority to, and the benefit of, U.S. Provisional Application No. 63/446,144, which was filed on Feb. 16, 2023, and is incorporated herein by reference in its entirety.

### FIELD OF THE INVENTION

The present invention relates generally to the field of massage stone heating and cooling devices. More specifically, the present invention relates to an improved device for heating and cooling massage stones. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in massage stone heating and cooling devices. Generally, the heating and cooling of massage stones is typically achieved using boiling water. However, using boiling water in this application is slow, messy, and dangerous. However, no alternative methods are readily available.

Typically, it is known to use a variety of devices to transfer heat energy to the body for therapeutic and relaxation purposes. In addition, it is known to use a variety of devices to simultaneously massage while delivering the heat. Different devices and methods from among those known vary in their performance, durability, versatility, efficiency and other characteristics. A common characteristic among most, however, is the ability to deliver heat through direct contact by positioning a heated device or heated stone on the area for which heat or massage is desired.

Accordingly, there is a demand for an improved massage stone heating and cooling device that heats and cools massage stones without the use of water. More particularly, there is a demand for a waterless heating and cooling device that comprises a lid and a digital temperature control.

Therefore, there exists a long felt need in the art for a massage stone heating and cooling device that provides users with an improved device for cooling and heating massage stones. There is also a long felt need in the art for a massage stone heating and cooling device that features a means for heating and cooling a massage stone without the use of water. Further, there is a long felt need in the art for a massage stone heating and cooling device that comprises a silicone mat insert that reduces noise and movement. Moreover, there is a long felt need in the art for a device that comprises a lid for more efficient temperature regulation. Further, there is a long felt need in the art for a massage stone heating and cooling device that comprises a digital temperature reader and controller to prevent the stones from getting too hot for the skin. Finally, there is a long felt need in the art for a massage stone heating and cooling device that provides a large surface area for heating or cooling more sizes of stones.

The subject matter disclosed and claimed herein, in one embodiment thereof, comprises a massage stone heating and cooling device for at least one massage stone. The device heats and cools massage stones via a waterless heating and cooling element. The massage stone heating and cooling device comprises a housing with a lid that offers more efficient temperature regulation. Further, the housing comprises a digital temperature reader and control which keeps the stones from getting too hot for the skin. The housing also comprises a silicone mat insert that reduces noise and movement. Additionally, the silicone mat insert offers a large surface area to accommodate more sizes of stones.

In this manner, the massage stone heating and cooling device of the present invention accomplishes all of the forgoing objectives and provides users with a device that heats and cools massage stones. The device is a housing with a waterless heating and cooling element and digital temperature control. The device can include a lid for temperature regulation.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a massage stone heating and cooling device for at least one massage stone. The device heats and cools massage stones via a waterless heating and cooling element. The massage stone heating and cooling device comprises a housing with a lid that offers more efficient temperature regulation. Further, the housing comprises a digital temperature reader and control which keeps the stones from getting too hot for the skin. The housing also comprises a silicone mat insert that reduces noise and movement.

In one embodiment, the massage stone heating and cooling device comprises a housing. The housing preferably has an open top. The housing can mate with a lid that can cover the opening of the housing to hold heat, cold, and massage stones therein. The housing is a heatable or coolable basin that is adapted to hold therein one or more massage stones or other suitable materials. The housing has a base portion (i.e., a floor), front and back walls, and opposing side walls which form an internal cavity for retaining a plurality of massage stones. Further, the housing comprises an electrical power cord, which can be plugged into an electrical wall outlet to provide current to an electric heating/cooling element housed in the base portion. The electric heating/cooling element is a waterless heating and cooling element. Upon delivering heat or cooling to the base portion, the massage stones in the housing are heated or cooled, as needed. Further, the massage stones can then be positioned into contact with body parts to deliver heat, cooling, massage, or skin conditioning therapy.

In one embodiment, the housing has a generally rectangular shape in the preferred embodiment, but is not limited to such a shape and may take on a variety of shapes and sizes as is known in the art.

In one embodiment, the lid secures the internal cavity and offers more efficient temperature regulation. In another embodiment, the lid preferably has a handle. The handle is preferably an integral handle formed on the lid, to prevent heat from escaping while the massage stones are heating/cooling or to prevent debris from inadvertently entering the chamber of the housing. The internal cavity of the housing may also have an inner surface of a metal, such as aluminum, which is known to have good heat transfer characteristics. In another embodiment, the housing comprises a silicone mat insert that reduces noise and movement when the device is in use. Specifically, the silicone mat insert is placed within the internal cavity and rests on the base portion or floor. The mat insert would be sized and shaped to fit within the internal cavity. Further, the plurality of massage stones would then be placed on the mat insert. Thus, during heating or cooling, the mat insert would reduce noise and movement of the massage stones and protect the stones within the device. The mat insert may also provide a break-resistant surface upon which massage stones placed or dropped into the base portion of the housing may impact. Additionally, the silicone mat insert offers a large surface area to accommodate more sizes of massage stones.

In another embodiment, the floor or base portion may include a series of ridges or other elements that form maximum surface contact area while maintaining an overall generally planar shape. The ridges can transfer heat for optimum heat transfer efficiency from the internal heating/cooling element, through the floor area. The mat insert may also comprise a series of ridges or other elements to provide for maximum surface contact area and optimum heat transfer efficiency from the internal heating/cooling element.

In one embodiment, the underside of the base portion or floor has a waterless heating and cooling element which can be a heat element wire that is preferably interwoven between and in contact with the underside of the base portion. This allows heat derived from electrical energy to be transferred to the floor and to the massage stones within the internal cavity. The heat element wire and the various electrical components used to operate the heating and cooling element are known to those of ordinary skill in the art. If desired, heat plates can be placed in contact with the underside of the floor instead of wires. Alternatively, the heat/cool element may comprise one or more heat elements on the outer surface of the base portion or floor, where the massage stones rest, instead of or in addition to providing one or more heat elements under the floor.

In one embodiment, the waterless heating and cooling element comprises a digital temperature reader and control which allows a user to set a predetermined temperature and keeps the massage stones from getting too hot or cool for the skin.

In one embodiment, the housing is generally hollow and can cover the internal components of the waterless heating and cooling element. The housing also has an aperture that allows the power cord to pass therethrough.

In one embodiment, the housing is manufactured of impact-resistant, water-resistant, and heat-resistant plastics or other suitable materials or combinations of materials. For example, the housing may be made from metal, plastic, ceramic, clay or other suitable materials. Further, the lid may be made of similar materials.

In one embodiment, the housing has an outer surface that has an operation switch positioned thereon. The operation switch controls on/off functions to activate or shut off heat/cool. The outer surface of the housing may also have an indicator light that may correspond to the “on” and “off” modes. If desired, one can add additional controls as are known in the art, such as temperature control, timers, and other similar features. Additional displays, including LED or LCD displays, may also be provided. Alternatively, the present invention may include a programmable controller or logic device in communication with a clock, a timer and/or an auto-shutoff component, and a temperature control. The outer surface of the housing may be hard-wired to an input/output terminal, which communicates with the controller, to send user input commands or to receive visual output signals, readouts, or audible signals. If desired, a remote control device which communicates with a control panel on the housing, may be used. Both the control panel and the remote control device can have push-button or touch-pad controls, and either can be used to operate the housing or to pre-program the housing.

In operation, after a sufficient period of time to heat the massage stones, the user may manually shut off the housing. In the event a user forgets to shut off the housing or if it stays on for an excessive period, the housing can be designed with an automatic shutoff feature to automatically shut itself off using any one of a variety of known safety shutoff technologies, as is known in the art. Thus, after heating/cooling for the preselected time duration, the housing will automatically shut-off or the user may manually override the automatic shut-off and shut the housing off selectively. Instead of a preselected time duration, the housing may also be programmed to shut-off when a predetermined temperature is reached, using temperature sensors and feedback controls of the type known to those generally skilled in the art.

Once the massage stones are heated or cooled as desired, the user removes the massage stones using tongs or by hand, and places them on parts of the body of the user (or another) for which heat/cool therapy is desired. At rest, the stones emit heat/cool to the user's body. If massage or skin therapy is desired, the massage stones may be moved manually against the user's body or skin to apply massaging movement and force, or to gently brush against skin surfaces. The types of massage stones used may vary depending upon such factors as heat retention, surface smoothness, shape and size, mineral qualities (i.e., salt content or aroma), availability, cost, etc., and other factors. In the preferred embodiment, it is desirable to use massage stones having smooth surfaces and sized to be easily handheld. Alternatively, the massage stones may be replaced by various types of heatable/coolable material objects. Such objects that may be used are, for example, glass, metal, ceramic, plastic, natural crystal, or composite. Such composites may include both or one of organic and inorganic materials. Additional materials having desirable heat conductive and surface properties may also be used.

In yet another embodiment, the massage stone heating and cooling device comprises a plurality of indicia.

In yet another embodiment, a method of heating or cooling massage stones without water is disclosed. The method includes the steps of providing a massage stone heating and cooling device comprising a housing with a lid and silicone mat insert, as well as a waterless heating and cooling element and digital temperature control. The method also comprises inserting at least one massage stone into the housing and securing the lid. Further, the method comprises plugging in the device via the electrical cord into a conventional wall outlet. The method also comprises setting the digital temperature control to a desired temperature setting. Finally, the method comprises heating or cooling the massage stone within the housing to the desired temperature for use.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one embodiment of the massage stone heating and cooling device of the present invention in accordance with the disclosed architecture;

FIG. 2 illustrates a side perspective view of one embodiment of the massage stone heating and cooling device of the present invention showing the digital temperature control in accordance with the disclosed architecture;

FIG. 3 illustrates a perspective view of one embodiment of the massage stone heating and cooling device of the present invention showing the attached lid in accordance with the disclosed architecture;

FIG. 4 illustrates a perspective view of one embodiment of the massage stone heating and cooling device of the present invention showing the lid removed in accordance with the disclosed architecture;

FIG. 5 illustrates a perspective view of one embodiment of the massage stone heating and cooling device of the present invention showing the different colors that are available in accordance with the disclosed architecture;

FIG. 6 illustrates a perspective view of one embodiment of the massage stone heating and cooling device of the present invention showing the different sizes and shapes that are available in accordance with the disclosed architecture;

FIG. 7 illustrates a perspective view of one embodiment of the massage stone heating and cooling device of the present invention showing the different colors and styles that are available in accordance with the disclosed architecture;

FIG. 8 illustrates a perspective view of one embodiment of the massage stone heating and cooling device of the present invention showing a variety of colors for the device in accordance with the disclosed architecture;

FIG. 9 illustrates a perspective view of one embodiment of the massage stone heating and cooling device of the present invention showing the removable silicone mat insert in accordance with the disclosed architecture;

FIG. 10 illustrates a perspective view of one embodiment of the massage stone heating and cooling device of the present invention showing the digital temperature control and reader in accordance with the disclosed architecture;

FIGS. 11A-C illustrate a perspective view of one embodiment of the massage stone heating and cooling device of the present invention showing the various components of the device in accordance with the disclosed architecture; and

FIG. 12 illustrates a flowchart showing the method of heating or cooling massage stones without water in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a massage stone heating and cooling device that provides users with an improved device for cooling and heating massage stones. There is also a long felt need in the art for a massage stone heating and cooling device that features a means for heating and cooling a massage stone without the use of water. Further, there is a long felt need in the art for a massage stone heating and cooling device that comprises a silicone mat insert that reduces noise and movement. Moreover, there is a long felt need in the art for a device that comprises a lid for more efficient temperature regulation. Further, there is a long felt need in the art for a massage stone heating and cooling device that comprises a digital temperature reader and controller to prevent the stones from getting too hot for the skin. Finally, there is a long felt need in the art for a massage stone heating and cooling device that provides a large surface area for heating or cooling more sizes of stones.

The present invention, in one exemplary embodiment, is a novel massage stone heating and cooling device for at least one massage stone. The device heats and cools massage stones via a waterless heating and cooling element. The massage stone heating and cooling device comprises a housing with a lid that offers more efficient temperature regulation. Further, the housing comprises a digital temperature reader and control which keeps the stones from getting too hot for the skin. The housing also comprises a silicone mat insert that reduces noise and movement. The present invention also includes a novel method of heating or cooling massage stones without water. The method includes the steps of providing a massage stone heating and cooling device comprising a housing with a lid and silicone mat insert, as well as a waterless heating and cooling element and digital temperature control. The method also comprises inserting at least one massage stone into the housing and securing the lid. Further, the method comprises plugging in the device via the electrical cord into a conventional wall outlet. The method also comprises setting the digital temperature control to a desired temperature setting. Finally, the method comprises heating or cooling the massage stone within the housing to the desired temperature for use.

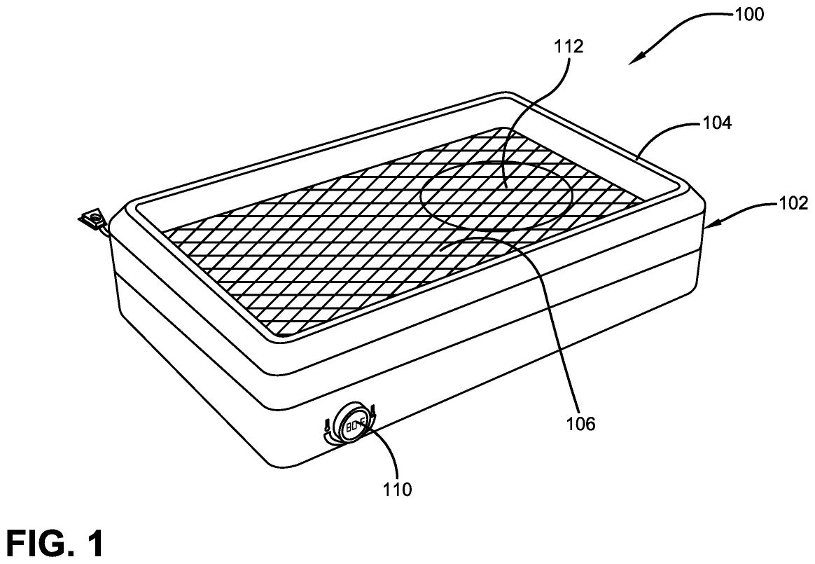

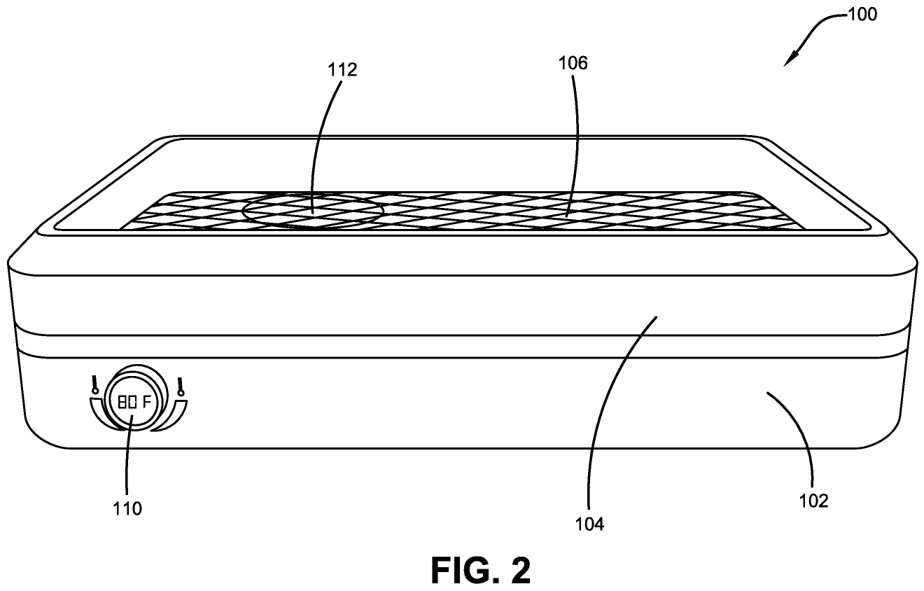

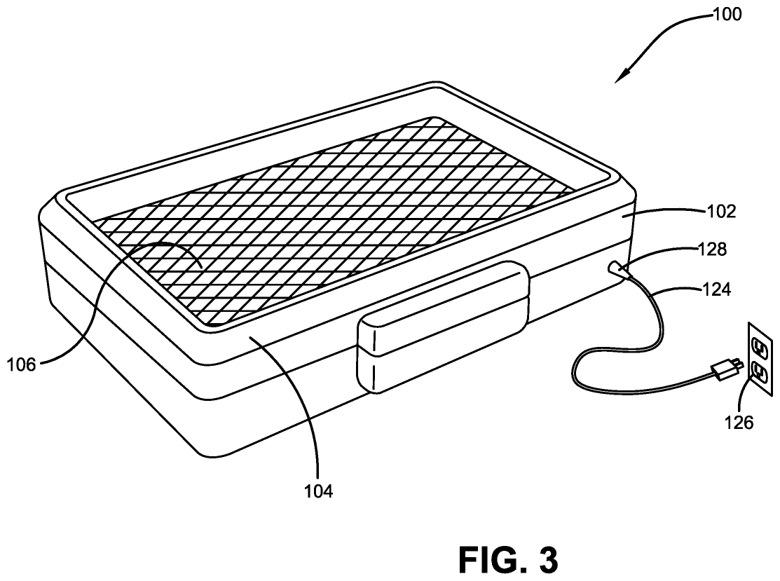

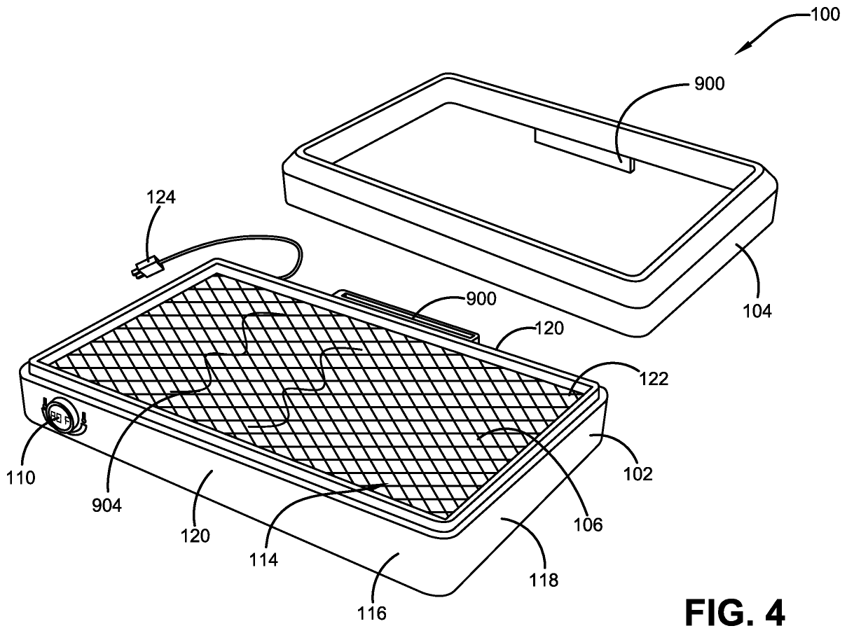

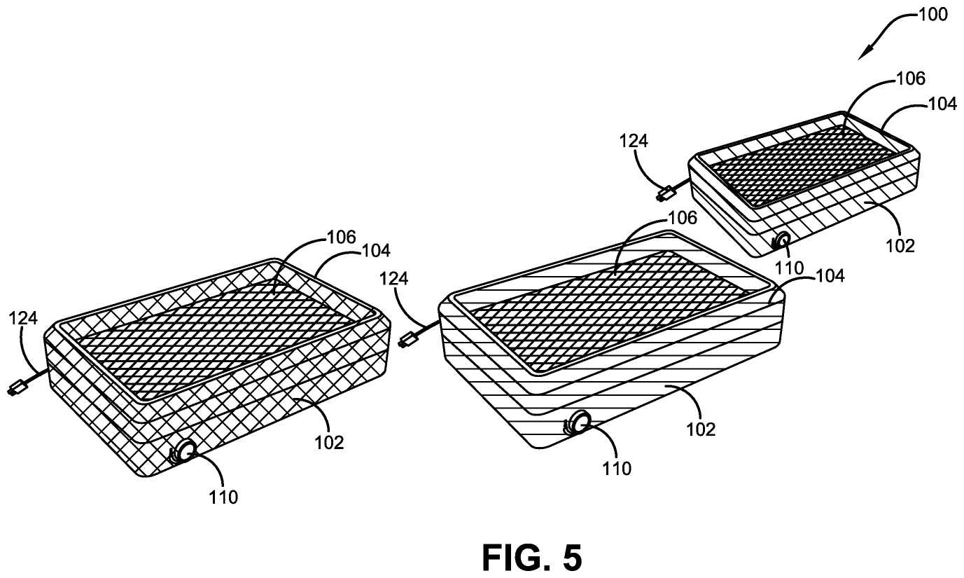

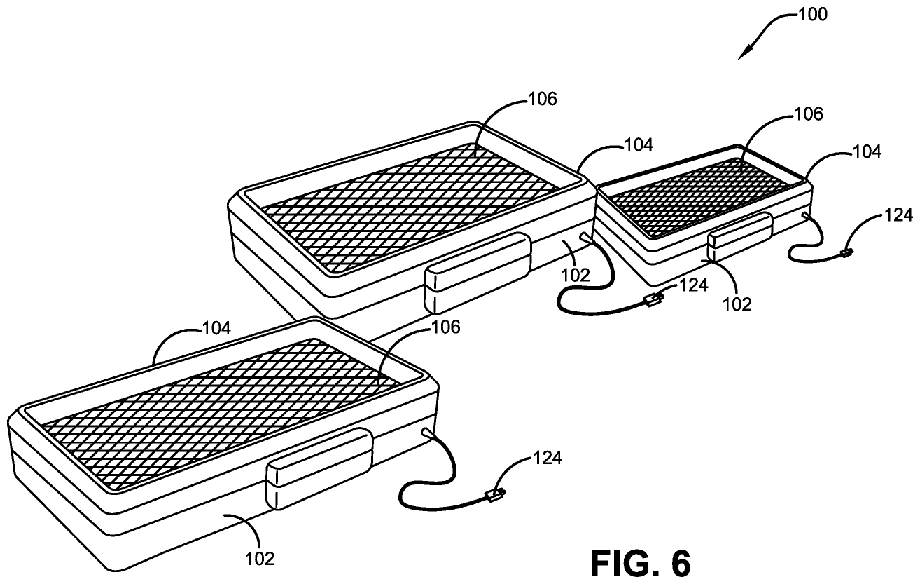

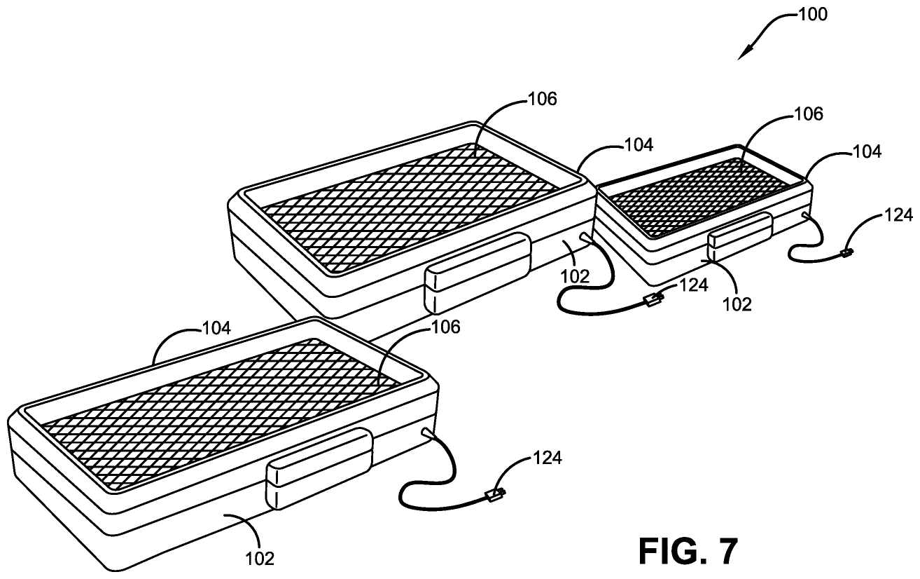

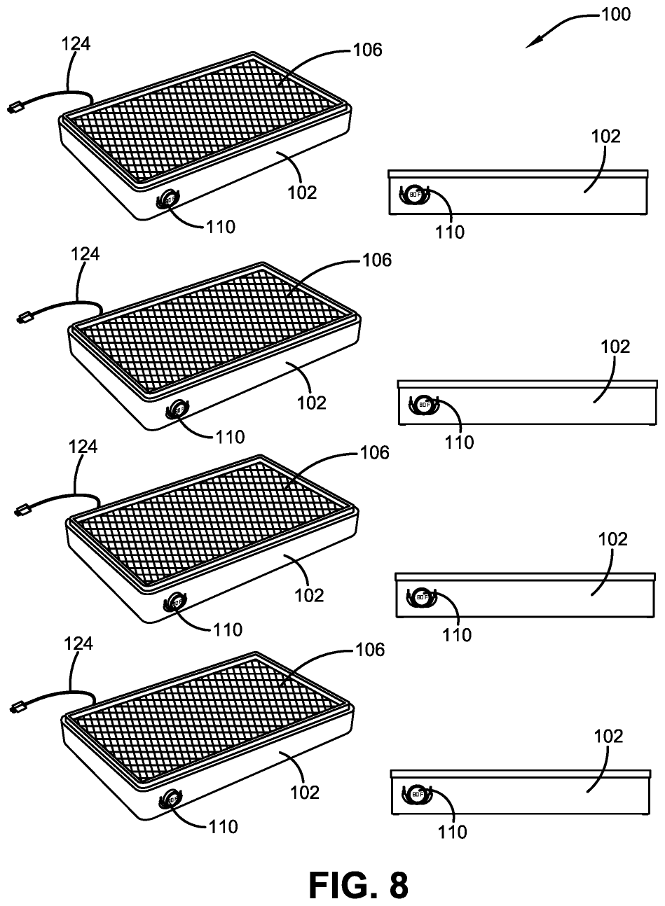

Referring initially to the drawings, FIGS. 1-8 illustrate a perspective view of one embodiment of the massage stone heating and cooling device 100 of the present invention. In the present embodiment, the massage stone heating and cooling device 100 is an improved massage stone heating and cooling device 100 that provides a user with a means for heating and cooling massage stones 112 without water. Specifically, the massage stone heating and cooling device 100 comprises a housing 102 with a lid component 104 and a silicone mat insert 106. The housing 102 also comprises a waterless heating and cooling element 108 and a digital temperature control 110, that keeps the stones 112 from getting too hot for a user's skin.

Generally, the housing 102 of the massage stone heating and cooling device 100 preferably has an open top 114. The housing 102 can mate with a lid component 104 that can cover the opening 114 of the housing 102 to hold heat, cold, and massage stones 112 therein. The housing 102 is a heatable or coolable basin that is adapted to hold therein one or more massage stones 112 or other suitable materials. The housing 102 has a base portion 116 (i.e., a floor), front and back walls 118, and opposing side walls 120 which form an internal cavity 122 for retaining a plurality of massage stones 112. Further, the housing 102 comprises an electrical power cord 124, which can be plugged into an electrical wall outlet 126 to provide current to an electric heating/cooling element 108 housed in the base portion 116. The electric heating/cooling element 108 is a waterless heating and cooling element. Upon delivering heat or cooling to the base portion 116, the massage stones 112 in the housing 102 are heated or cooled, as needed. Further, the massage stones 112 can then be positioned into contact with body parts to deliver heat, cooling, massage, or skin conditioning therapy. In one embodiment, the housing 102 can be powered by batteries.

Additionally, the housing 102 is generally hollow and can cover the internal components of the waterless heating and cooling element 108. The housing 102 also has an aperture 128 that allows the power cord 124 to pass therethrough.

Furthermore, the housing 102 has a generally rectangular shape in the preferred embodiment, but is not limited to such a shape and may take on a variety of shapes and sizes as is known in the art, depending on the needs and/or wants of a user. Additionally, the housing 102 can be any suitable color or style as is known in the art, and can be decorated with various aesthetic designs and embellishments, to match a user's decor and/or mood.

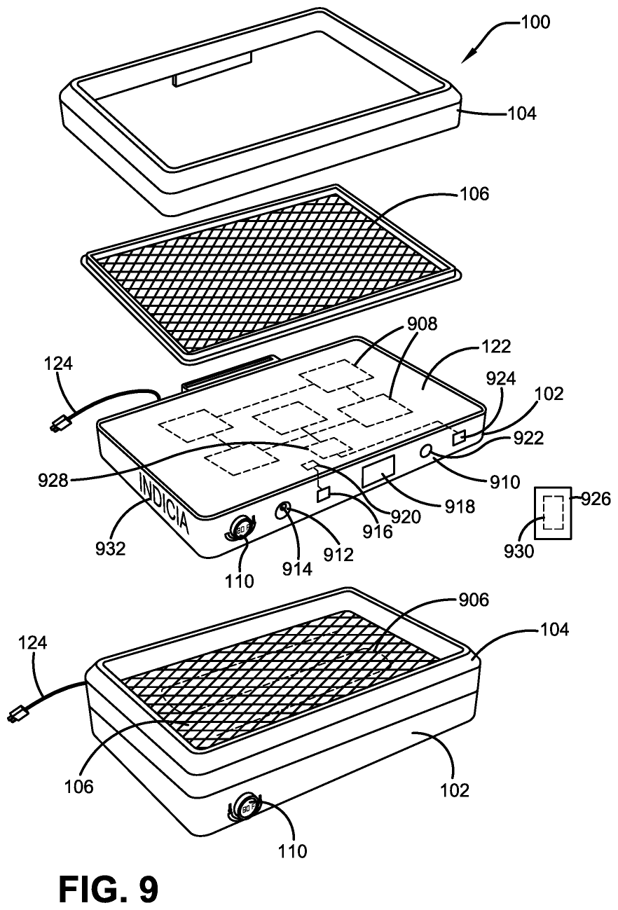

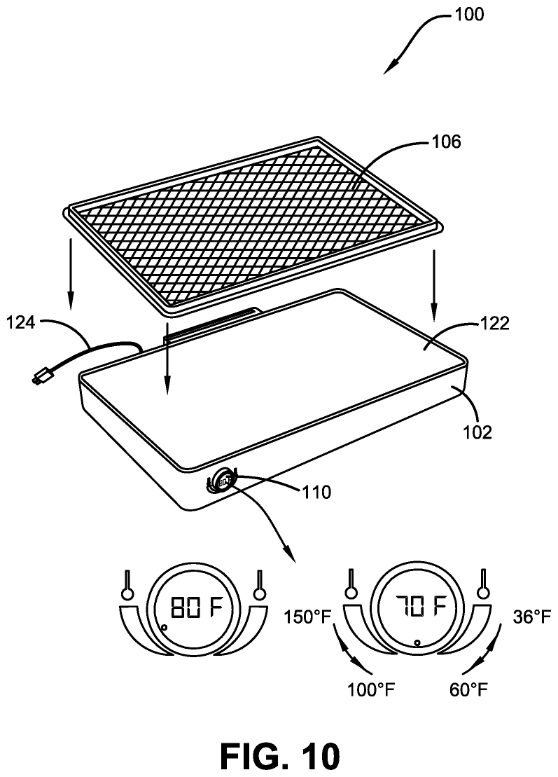

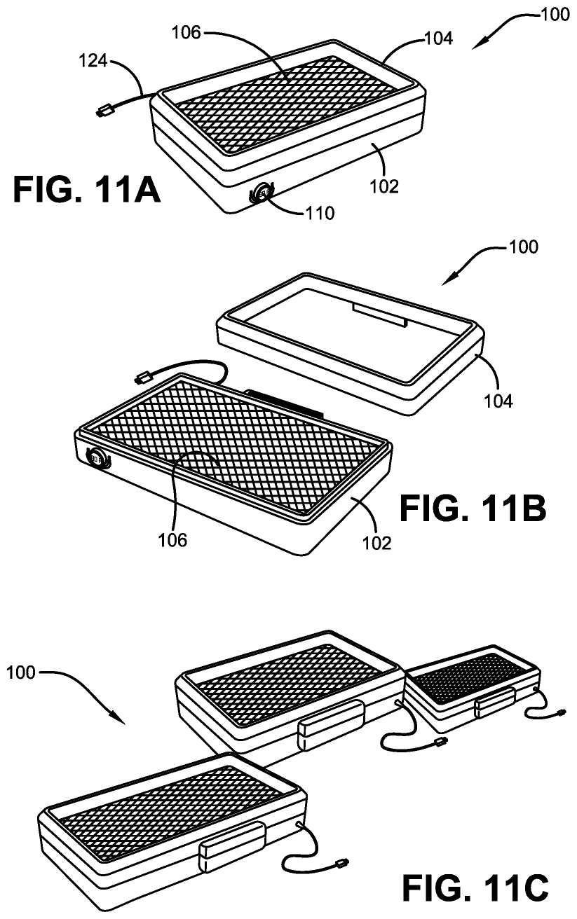

As shown in FIGS. 9-11, the lid component 104 is utilized to secure the internal cavity 122 and offers more efficient temperature regulation. In another embodiment, the lid component 104 preferably has a handle 900. The handle 900 can be any suitable shape and size as is known in the art. The handle 900 is preferably an integral handle formed on the lid 104, to prevent heat from escaping while the massage stones 112 are heating/cooling or to prevent debris from inadvertently entering the chamber of the housing 102. The internal cavity 122 of the housing 102 may also have an inner surface 902 of a metal, such as aluminum, which is known to have good heat transfer characteristics.

Furthermore, the housing 102 comprises a silicone mat insert 106 that reduces noise and movement when the device 100 is in use. Specifically, the silicone mat insert 106 is placed within the internal cavity 122 and rests on the base portion 116 or floor. The mat insert 106 would be sized and shaped to fit within the internal cavity 122. Further, the plurality of massage stones 112 would then be placed on the mat insert 106, during use. Thus, during heating or cooling, the mat insert 106 would reduce noise and movement of the massage stones 112 and protect the stones 112 within the device 100. The mat insert 106 may also provide a break-resistant surface upon which massage stones 112 placed or dropped into the base portion 116 of the housing 102 may impact. Additionally, the silicone mat insert 106 offers a large surface area to accommodate more sizes and amounts of massage stones 112.

In another embodiment, the floor or base portion 116 may include a series of ridges 904 or other elements that form maximum surface contact area while maintaining an overall generally planar shape. The ridges 904 can transfer heat for optimum heat transfer efficiency from the internal heating/cooling element 108, through the floor area 116. Further, the mat insert 106 may also comprise a series of ridges 904 or other elements to provide for maximum surface contact area and optimum heat transfer efficiency from the internal heating/cooling element 108.

In one embodiment, the underside of the base portion or floor 116 has a waterless heating and cooling element 108 which can be a heat element wire 906 that is preferably interwoven between and in contact with the underside of the base portion 116. This allows heat derived from electrical energy to be transferred to the floor 116 and to the massage stones 112 within the internal cavity 122. The heat element wire 906 and the various electrical components used to operate the heating and cooling element 108 are known to those of ordinary skill in the art. If desired, heat plates 908 can be placed in contact with the underside of the floor 116 instead of wires 906. Alternatively, the heat/cool element 108 may comprise one or more heat elements 906 or 908 on the outer surface of the base portion or floor 116, where the massage stones 112 rest, instead of or in addition to providing one or more heat elements 906 or 908 under the floor 116.

Furthermore, the waterless heating and cooling element 108 comprises a digital temperature reader and control 110 which allows a user to set a predetermined temperature and keeps the massage stones 112 from getting too hot or cool for a user's skin.

Additionally, the housing 102 has an outer surface 910 that has an operation switch 912 positioned thereon. The operation switch 912 controls on/off functions to activate or shut off heat/cool. The outer surface 910 of the housing 102 may also have an indicator light 914 that may correspond to the “on” and “off” modes. If desired, one can add additional controls as are known in the art, such as temperature control, timers, and other similar features. Additional displays 918, including LED or LCD displays, may also be provided. Alternatively, the present invention may include a programmable controller or logic device 920 in communication with a timer 916, an auto-shutoff component 922, and a temperature control 110. The outer surface 910 of the housing 102 may be hard-wired to an input/output terminal 924, which communicates with the controller 920, to send user input commands or to receive visual output signals, readouts, or audible signals. If desired, a remote control device 926 which communicates with a control panel 928 on the housing 102, may be used. Both the control panel 928 and the remote control device 926 can have push-button or touch-pad controls 930, and either can be used to operate the housing 102 or to pre-program the housing 102.

In operation, after a sufficient period of time to heat the massage stones 112, the user may manually shut off the housing 102. In the event a user forgets to shut off the housing 102 or if it stays on for an excessive period of time, the housing 102 can be designed with an automatic shutoff feature to automatically shut itself off using any one of a variety of known safety shutoff technologies, as is known in the art. Thus, after heating/cooling for the preselected time duration, the housing 102 will automatically shut-off or the user may manually override the automatic shut-off 922 and shut the housing 102 off selectively. Instead of a preselected time duration, the housing 102 may also be programmed to shut-off when a predetermined temperature is reached, using temperature sensors and feedback controls of the type known to those generally skilled in the art.

Once the massage stones 112 are heated or cooled as desired, the user removes the massage stones 112 using tongs or by hand, and places them on parts of the body of the user (or another) for which heat/cool therapy is desired. At rest, the stones 112 emit heat/cool to the user's body. If massage or skin therapy is desired, the massage stones 112 may be moved manually against the user's body or skin to apply massaging movement and force, or to gently brush against skin surfaces. The types of massage stones 112 used may vary depending upon such factors as heat retention, surface smoothness, shape and size, mineral qualities (i.e., salt content or aroma), availability, cost, etc., and other factors. In the preferred embodiment, it is desirable to use massage stones 112 having smooth surfaces and sized to be easily handheld. Alternatively, the massage stones 112 may be replaced by various types of heatable/coolable material objects. Such objects that may be used are, for example, glass, metal, ceramic, plastic, natural crystal, or composite. Such composites may include both or one of organic and inorganic materials. Additional materials having desirable heat conductive and surface properties may also be used.

In one embodiment, the housing 102 is manufactured of impact-resistant, water-resistant, and heat-resistant plastics or other suitable materials or combinations of materials. For example, the housing 102 may be made from metal, plastic, ceramic, clay or other suitable materials as is known in the art. Further, the lid component 104 may be made of similar materials as is known in the art.

In yet another embodiment, the massage stone heating and cooling device 100 comprises a plurality of indicia 932. The housing 102 of the device 100 may include advertising, a trademark, or other letters, designs, or characters, printed, painted, stamped, or integrated into the housing 102, or any other indicia 932 as is known in the art. Specifically, any suitable indicia 932 as is known in the art can be included, such as but not limited to, patterns, logos, emblems, images, symbols, designs, letters, words, characters, animals, advertisements, brands, etc., that may or may not be massage stone, heating/cooling, or brand related.

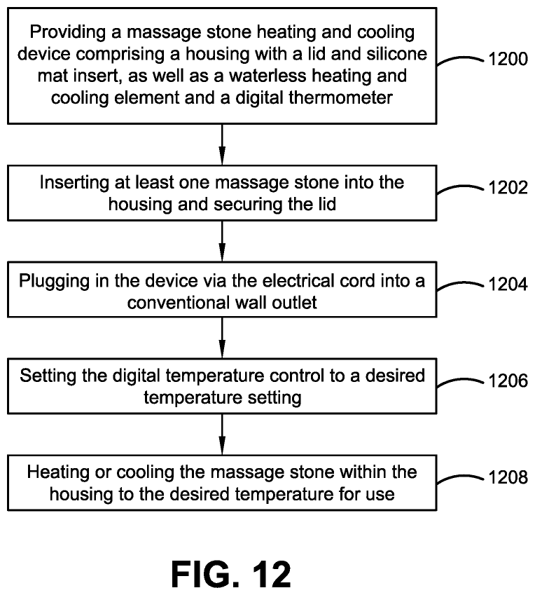

FIG. 12 illustrates a flowchart of the method of heating or cooling massage stones without water. The method includes the steps of at 1200 providing a massage stone heating and cooling device comprising a housing with a lid and silicone mat insert, as well as a waterless heating and cooling element and digital temperature control. The method also comprises at 1202 inserting at least one massage stone into the housing and securing the lid. Further, the method comprises at 1204, plugging in the device via the electrical cord into a conventional wall outlet. The method also comprises at 1206, setting the digital temperature control to a desired temperature setting. Finally, the method comprises at 1208, heating or cooling the massage stone within the housing to the desired temperature for use.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “massage stone heating and cooling device”, “massage stone device”, “heating and cooling device”, and “device” are interchangeable and refer to the massage stone heating and cooling device 100 of the present invention.

Notwithstanding the forgoing, the massage stone heating and cooling device 100 of the present invention can be of any suitable size and configuration as is known in the art without affecting the overall concept of the invention, provided that it accomplishes the above stated objectives. One of ordinary skill in the art will appreciate that the massage stone heating and cooling device 100 as shown in FIGS. 1-12 is for illustrative purposes only, and that many other sizes and shapes of the massage stone heating and cooling device 100 are well within the scope of the present disclosure. Although the dimensions of the massage stone heating and cooling device 100 are important design parameters for user convenience, the massage stone heating and cooling device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A massage stone heating and cooling device that provides a user with a means for heating and cooling massage stones without water, the massage stone heating and cooling device comprising:
a housing;
a lid component; and
a waterless heating and cooling element;
wherein at least one massage stone is placed within the housing and the lid component is secured; and
further wherein the waterless heating and cooling element heats or cools the at least one massage stone within the housing for use.

2. The massage stone heating and cooling device of claim 1, wherein the housing has an open top which is secured via the lid component.

3. The massage stone heating and cooling device of claim 2, wherein the housing has a base portion, front and back walls, and opposing side walls which form an internal cavity for retaining the at least one massage stone.

4. The massage stone heating and cooling device of claim 3, wherein the housing comprises an electrical power cord, which can be plugged into an electrical wall outlet to provide current to the waterless heating and cooling element housed in the base portion.

5. The massage stone heating and cooling device of claim 4, wherein the lid component has a handle.

6. The massage stone heating and cooling device of claim 5, wherein the internal cavity of the housing comprises an inner surface of a metal.

7. The massage stone heating and cooling device of claim 6, wherein a mat insert is positioned within the internal cavity and rests on the base portion, and the at least one massage stone would then be placed on the mat insert during use.

8. The massage stone heating and cooling device of claim 7, wherein the mat insert comprises a series of ridges for maximum surface contact area.

9. The massage stone heating and cooling device of claim 8, wherein the waterless heating and cooling element is a heat element wire positioned above or below the base portion.

10. The massage stone heating and cooling device of claim 8, wherein the waterless heating and cooling element is a heat plate positioned above or below the base portion.

11. The massage stone heating and cooling device of claim 8, wherein the waterless heating and cooling element comprises a digital temperature reader and control which allows a user to set a predetermined temperature and keep the at least one massage stone from getting too hot or cool for a user's skin.

12. The massage stone heating and cooling device of claim 11, wherein the housing has an operation switch and an indicator light for turning off and on the massage stone heating and cooling device.

13. The massage stone heating and cooling device of claim 12, wherein the housing comprises a programmable controller in communication with a timer, an auto-shutoff component, and the digital temperature reader and control.

14. The massage stone heating and cooling device of claim 13 further comprising a remote control.

15. A massage stone heating and cooling device that provides a user with a means for heating and cooling massage stones without water, the massage stone heating and cooling device comprising:
a housing with an operation switch and an indicator light;
a lid component with a handle; and
a waterless heating and cooling element that comprises a digital temperature reader and control which allows a user to set a predetermined temperature; and
wherein the housing has an open top which is secured via the lid component;
wherein the housing has a base portion, front and back walls, and opposing side walls which form an internal cavity for retaining at least one massage stone;
wherein the housing comprises an electrical power cord, which can be plugged into an electrical wall outlet to provide current to the waterless heating and cooling element housed in the base portion;
wherein the internal cavity of the housing comprises an inner surface of a metal;
wherein a mat insert is positioned within the internal cavity and rests on the base portion, and the at least one massage stone would then be placed on the mat insert during use;
wherein the waterless heating and cooling element heats or cools the at least one massage stone within the housing; and
further wherein the at least one massage stone is then removed from the housing and placed on a user's body to emit heat or cool.

16. The massage stone heating and cooling device of claim 15 further comprising a plurality of indicia.

17. The massage stone heating and cooling device of claim 15, wherein the mat insert comprises a series of ridges for maximum surface contact area.

18. The massage stone heating and cooling device of claim 15, wherein the waterless heating and cooling element is a heat element wire positioned above or below the base portion.

19. The massage stone heating and cooling device of claim 15, wherein the waterless heating and cooling element is a heat plate positioned above or below the base portion.

20. A method of heating or cooling massage stones without water, the method comprising the following steps:
providing a massage stone heating and cooling device comprising a housing with a lid and silicone mat insert, as well as a waterless heating and cooling element and digital temperature control;
inserting at least one massage stone into the housing and securing the lid;
plugging in the device via the electrical cord into a conventional wall outlet;
setting the digital temperature control to a desired temperature setting; and
heating or cooling the massage stone within the housing to the desired temperature for use.
